(19) United States
(12) Patent Application Publication
(10) Pub. No.: US 2018/0251824 A1
(43) Pub. Date: Sep. 6, 2018

(54) IN VITRO GENOTOXICITY ASSAY USING A TRANSCRIPTOMIC BIOMARKER WITH DIRECT DIGITAL COUNTING

(71) Applicant: GEORGETOWN UNIVERSITY, Washington, DC (US)

(72) Inventors: Albert J. Fornace, JR., Washington, DC (US); Heng-Hong Li, Rockville, MD (US)

(73) Assignee: GEORGETOWN UNIVERSITY, Washington, DC (US)

(21) Appl. No.: 15/909,198

(22) Filed: Mar. 1, 2018

Related U.S. Application Data
(60) Provisional application No. 62/465,591, filed on Mar. 1, 2017.

(51) Int. Cl. C12Q 1/6827; G01N 33/50

(52) U.S. Cl. CPC C12Q 1/6827; C12Q 2600/142; C12Q 2600/156; G01N 33/5014

(57) ABSTRACT

Provided herein is a method of detecting a DNA damage-inducing (DDI) agent using a transcriptomic biomarker, wherein the biomarker comprises at least 63 genes, and direct digital counting.

### CROSS-REFERENCE TO PRIORITY APPLICATIONS

This application claims priority to U.S. Provisional Application No. 62/465,591, filed Mar. 1, 2017, the entirety of which is incorporated by reference herein.

### STATEMENT REGARDING FEDERALLY FUNDED RESEARCH

This invention was made with Government Support under grant numbers R43-ES026473-01, R43-ES026473 and 1RO1-ES020750 awarded by the National Institute of Environmental Health Sciences. The Government has certain rights in the invention.

### BACKGROUND

Interpretation of positive genotoxicity findings in the in vitro testing battery is a major challenge to both industry and regulatory agencies. These tests have high sensitivity but suffer from low specificity, leading to high rates of irrelevant positive findings (i.e., positive results in vitro that are not relevant or reproduced in vivo). Genotoxicity represented by chromosome damage and mutations in DNA is considered to be the hallmark of carcinogenic risk. The standard genotoxicity assays, especially in the case of in vitro chromosome aberration assays, have a high ‘false’ positive rate, which results in costly and time consuming follow up assays that increase the cost of drug development and chemical safety assessment. Hence, gaining insight into genotoxic mechanisms and distinguishing these irrelevant (false) positive genotoxicity findings caused by nongenotoxic mechanisms is of great value.

### SUMMARY

Provided herein is a method of identifying a test agent as a DNA damaging agent (DDI) agent or a non-DDI (NDDI) agent. The method comprises contacting a cell with a test agent; lysing the cell; obtaining a gene expression profile by determining the gene expression levels of at least 63 genes (e.g., 64 genes) from Table 1 in the cell lysate, wherein the gene expression levels of the 64 genes are determined using direct digital counting. The gene expression profile obtained is compared to a gene expression profile for each of a plurality of training samples that have been classified in a subtype of DDI agents or a subtype of non-DDI agents, wherein the gene expression profile for each of the plurality of training samples is based on gene expression of the genes. A supervised algorithm is used to construct centroids for each of the DDI and non-DDI agent subtypes in the training set and the distance of the gene expression profile obtained is calculated to each of the centroids. The test agent is identified as a DDI agent or a non-DDI agent based upon the nearest centroid.

### BRIEF DESCRIPTION OF THE DRAWINGS

FIG. 1 shows a diagram of an in vitro chromosome damage assay.

FIG. 2 shows cell viability at selected doses for each compound, measured using an MTT assay. Cell viability 24 h after start of treatment varied and did not correlate with genotoxicity.

FIG. 3A and FIG. 3B show genotoxicity prediction using the TGx-DD1 (also known as TGx-28.65) transcriptomic biomarker. FIG. 3A shows the results for Class 1 and FIG. 3B shows the results for Class 5 as representative transcriptional responses for dose-optimization indicator genes, ATF3, CDKN1A and GADD45A, measured by qRT-PCR. The ratio designates the relative change in gene expression compared to vehicle-treated control cells. Results are shown for the concentrations selected for subsequent microarray experiments.

FIG. 4 shows concentration range-finding studies guided by assessing expression of three stress responsive genes, CDKN1A, GADD45A and ATF3, using qRT-PCR. To enable comparison of transcriptome profiles across the whole set of agents at a single concentration per chemical and to establish a strategy for setting concentrations for new test compounds, a qRT-PCR stress gene panel expression protocol was followed. The ratio designates the relative change in gene expression compared to vehicle-treated control cells. Briefly, cells were treated over a broad concentration range, and results are shown for the concentrations selected for subsequent microarray experiments.

FIG. 5A, FIG. 5B, FIG. 5C, FIG. 5D, and FIG. 5E show results of a DDI prediction method using TGx-DDI, namely principal component analysis (PCA) analysis, which was performed along with 2-dimensional clustering (2DC), for microarray data of the validation chemicals from five general classes. Agents in light gray circles and dark gray circles are the DDI and non-DDI agents, respectively, from the original training set; agents in triangles are the validation chemicals for each of the five classes. In brief, a chemical clustering with the DDI branch in 2DC plot is called DDI, and vice versa for non-DDI agents. In PCA plot, chemicals with a negative first principal component (PC1) are classified as DDI, and with a positive PC1 are classified as non-DDI. The plots of class 1 to 5 are displayed in FIGS. 5A-5E, respectively. The results show that all validation chemicals in class 5 except one are classified as non-DDI. These chemicals are known to have irrelevant positive results in chromosomal aberration assay.

FIG. 6 shows performance of TGx-DDI with Nanostring nCounter, which was compared with TGx-DDI by microarray. DDI prediction was performed based on nCounter results using 2DC method as shown in the heatmaps. Log 2-fold-change correlation of 65 genes as measured by Nanostring nCounter and microarray is shown. A linear fit yields a correlation coefficient of 0.91.

FIG. 7A, FIG. 7B, FIG. 7C, and FIG. 7D_show PCA analysis of nCounter data of TGx-DDI for the test chemicals in Class 1, 2, 4, and 5, respectively. In brief, a chemical clustering with the DDI branch in 2DC plot is called DDI, and vice versa for non-DDI agents. In PCA plot, chemicals with a negative first principal component (PC1) are classified as DDI, and with a positive PC1 are classified as non-DDI.

FIG. 7E shows results for chemicals that require metabolic activation. Consistent with the results from microarray profiling (FIG. 5), the nCounter results show that all validation chemicals in class 5, except one, are classified as non-DDI.

FIG. 8 shows representative log 2-fold-change correlation of 64 genes in total RNA and cell lysates in a comparison of nCounter results using cell lysate and total RNA methods from cells treated with bleomycin. The correlation between results using total RNA and cell lysates was analyzed; the R square calculated based on linear regression ranges from 0.90 to 0.96 for each cell concentration. Shown here is the comparison of total RNA and cell lysate with a concentration of 2000 cells/μl.

FIG. 9A and FIG. 9B show a proposed workflow for applying the TGx-DDI biomarker for genotoxicity assessment of a drug (FIG. 9A) or a chemical (FIG. 9B).

### DETAILED DESCRIPTION

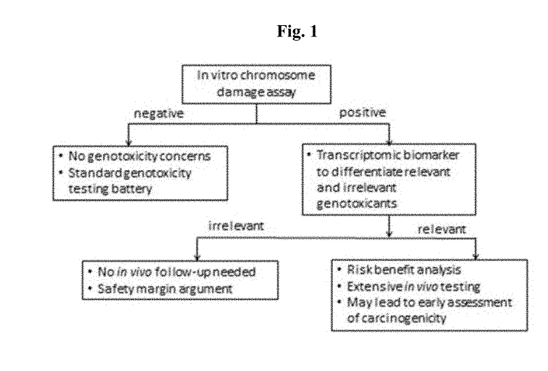

Provided herein is a highly specific biomarker panel that can be used to identify DNA damage-inducing agents (i.e., agents that cause adducts or other DNA lesions such as DNA strand breaks that could result in gene mutations or chromosome structural aberrations through either direct or certain indirect DNA-damage-like processes that are relevant to in vivo genotoxicity outcomes) using a transcriptomics approach employing a direct digital counting technology to achieve high levels of precision, linearity, and reproducibility in measuring the expression levels of 55-65 genes, for example, 63-65 genes in a transcriptomic biomarker, simultaneously. Thus provided herein is a method of detecting a DNA damage-inducing (DDI) agent using a transcriptomic biomarker, wherein the biomarker comprises 55, 56, 57, 58, 59, 60, 61, 62, 63 or 64 genes from Table 1. In some examples, the biomarker comprises 63-65 genes, for example, 64 genes, from Table 1. The method is optionally used as a follow-up to a positive chromosome damage assay in mammalian cells to provide mechanistic insights by differentiating relevant from irrelevant in vitro genotoxicants, as shown in FIG. 1.

The biomarker used in the methods described herein comprises a gene expression signature of specific genes involved in genotoxic stress responses. In some examples, the biomarker, designated TGx-DDI comprises 64 genes with a bias towards genes that are responsive to DNA damage. More specifically, the transcriptomic biomarker TGx-DDI is capable of differentiating relevant from irrelevant in vitro DDI agents by measurement of transcriptional DNA damage response. The biomarker works with a diverse panel of chemicals having a variety of mechanisms of action, allowing for accurate high-throughput genotoxicity screening with marked savings in cost compared to animal testing, which is often needed to address concerns about irrelevant positive tests with current toxicology approaches. The method provided herein provides significant benefits and commercial value, in comparison to the current genotoxicity battery.

Provided herein is a method of identifying a test agent as a DNA damaging agent (DDI) agent or a non-DDI (NDDI) agent comprising (a) contacting a cell with a test agent; (b) lysing the cell; (c) obtaining a gene expression profile by determining the gene expression levels of at least 63 genes (e.g. 64 genes) from Table 1 in the cell lysate, wherein the gene expression levels of 64 genes are determined using direct digital counting; and (d) comparing the gene expression profile obtained in step c) to a gene expression profile for each of a plurality of training samples that have been classified in a subset of DDI agents or a subset of non-DDI agents, wherein the gene expression profile for each of the plurality of training samples is based on gene expression of the genes from Table 1, and wherein a supervised algorithm was used to construct centroids for each of the DDI and non-DDI agent subsets in the training set; (e) calculating the distance of the gene expression profile obtained in step (b) to each of the centroids; and (f) identifying the test agent as a DDI agent or a NDDI agent based upon the nearest centroid.

In the methods provided herein, the test agent can be a chemical, a small or large molecule (organic or inorganic), a drug, a peptide, a cDNA, an antibody, an aptamer, a morpholino, a triple helix molecule, an siRNA, a shRNA, an miRNA, an antisense RNA, or a ribozyme. The agent(s) to be tested are contacted with the cell for a sufficient time period to allow the test agent to exert its effects, if any, on the cell. This time can vary depending on the test agent, the cell and other factors. For example, the test agent can be contacted with the cell for about two, four, six, eight, ten hours or more. In some examples, contact is performed for about four hours. Optionally, the concentration of the test agent is optimized, for example, by conducting dose response studies prior to contacting the cells with the test agent. Optionally, multiple concentrations of the test agent are tested to obtain does response results. One of skill in the art would know how to determine a concentration of the test agent that triggers a measureable transcriptional response. See, for example, Li et al., “Development of toxicogenomics signature for genotoxicity using a dose-optimization and informatics strategy for human cells,” Environ. Mol. Mutagen. 56(6): 505-519 (2015).

In the methods provided herein, the cell is lysed prior to obtaining an expression profile for at least 63 genes (e.g. 64 genes) from Table 1. Methods for lysing cells are known in the art and include, but are not limited to, sonication, freeze-thaw lysis, thermolysis, detergent lysis, and enzymatic lysis. Optionally, RNA can be isolated from the lysed cells prior to using the isolated RNA to obtaining an expression profile for the genes from Table 1.

Table 1 shows the genes that constitute TGx-DDI. A description and GenBank Accession No. is also provided for each gene. Optionally, in some methods, the gene expression level of USP41 is not included in the expression profile obtained from a cell contacted with a test agent.

TABLE 1

| GeneSymbol | Description | Genbank Accession |
|---|---|---|
| ACTA2 | Homo sapiens actin, alpha 2, smooth muscle, aorta (ACTA2), transcript | NM_001613 |
| | variant 2, mRNA [NM_001613] | |
| AEN | Homo sapiens apoptosis enhancing nuclease (AEN), mRNA [NM_022767] | NM_022767 |
| ARRDC4 | Homo sapiens arrestin domain containing 4 (ARRDC4), mRNA [NM_183376] | NM_183376 |
| B3GNT2 | Homo sapiens UDP-GlcNAc:betaGal beta-1,3-N- | NM_006577 |
| | acetylglucosaminyltransferase 2 (B3GNT2), mRNA [NM_006577] | |
| BLOC1S2 | Homo sapiens biogenesis of lysosomal organelles complex-1, subunit 2 | NM_001001342 |
| | (BLOC1S2), transcript variant 2, mRNA [NM_001001342] | |
| BRMS1L | Homo sapiens breast cancer metastasis-suppressor 1-like (BRMS1L), mRNA | NM_032352 |
| | [NM_032352] | |
| BTG2 | Homo sapiens BTG family, member 2 (BTG2), mRNA [NM_006763] | NM_006763 |
| C12orf5 | Homo sapiens chromosome 12 open reading frame 5 (C12orf5), mRNA | NM_020375 |
| | [NM_020375] | |
| CBLB | Homo sapiens Cas-Br-M (murine) ecotropic retroviral transforming sequence | NM_170662 |
| | b (CBLB), mRNA [NM_170662] | |
| CCP110 | Homo sapiens centriolar coiled coil protein 110 kDa (CCP110), transcript | NM_014711 |
| | variant 2, mRNA [NM_014711] | |
| CDKN1A | Homo sapiens cyclin-dependent kinase inhibitor 1A (p21, Cip1) (CDKN1A), | NM_078467 |
| | transcript variant 2, mRNA [NM_078467] | |
| CEBPD | Homo sapiens CCAAT/enhancer binding protein (C/EBP), delta (CEBPD), | NM_005195 |
| | mRNA [NM_005195] | |
| CENPE | Homo sapiens centromere protein E, 312 kDa (CENPE), mRNA [NM_001813] | NM_001813 |
| COIL | Homo sapiens coilin (COIL), mRNA [NM_004645] | NM_004645 |
| DAAM1 | Homo sapiens dishevelled associated activator of morphogenesis 1 (DAAM1), | NM_014992 |
| | mRNA [NM_014992] | |
| DCP1B | Homo sapiens DCP1 decapping enzyme homolog B (S. cerevisiae) (DCP1B), | NM_152640 |
| | mRNA [NM_152640] | |
| DDB2 | Homo sapiens damage-specific DNA binding protein 2, 48 kDa (DDB2), mRNA | NM_000107 |
| | [NM_000107] | |
| DUSP14 | Homo sapiens dual specificity phosphatase 14 (DUSP14), mRNA | NM_007026 |
| | [NM_007026] | |
| E2F7 | Homo sapiens E2F transcription factor 7 (E2F7), mRNA [NM_203394] | NM_203394 |
| E2F8 | Homo sapiens E2F transcription factor 8 (E2F8), mRNA [NM_024680] | NM_024680 |
| EI24 | Homo sapiens etoposide induced 2.4 mRNA (EI24), transcript variant 1, | NM_004879 |
| | mRNA [NM_004879] | |
| FAM123B | Homo sapiens family with sequence similarity 123B (FAM123B), mRNA | NM_152424 |
| | [NM_152424] | |
| FBXO22 | Homo sapiens F-box protein 22 (FBXO22), transcript variant 1, mRNA | NM_147188///NM_012170 |
| | [NM_147188]///Homo sapiens F-box protein 22 (FBXO22), transcript variant 2, | |
| | mRNA [NM_012170] | |
| GADD45A | Homo sapiens growth arrest and DNA-damage-inducible, alpha (GADD45A), | NM_001924 |
| | transcript variant 1, mRNA [NM_001924] | |
| GXYLT1 | Homo sapiens glucoside xylosyltransferase 1 (GXYLT1), transcript variant 1, | NM_173601 |
| | mRNA [NM_173601] | |
| HIST1H1E | Homo sapiens histone cluster 1, H1e (HIST1H1E), mRNA [NM_005321] | NM_005321 |
| HIST1H2BB | Homo sapiens histone cluster 1, H2bb (HIST1H2BB), mRNA [NM_021062] | NM_021062 |
| HIST1H2BC | Homo sapiens histone cluster 1, H2bc (HIST1H2BC), mRNA [NM_003526] | NM_003526 |
| HIST1H2BG | Homo sapiens histone cluster 1, H2bg (HIST1H2BG), mRNA [NM_003518] | NM_003518 |
| HIST1H2BI | Homo sapiens histone cluster 1, H2bi (HIST1H2BI), mRNA [NM_003525] | NM_003525 |
| HIST1H2BM | Homo sapiens histone cluster 1, H2bm (HIST1H2BM), mRNA [NM_003521] | NM_003521 |
| HIST1H2BN | Homo sapiens histone cluster 1, H2bn (HIST1H2BN), mRNA [NM_003520] | NM_003520 |
| HIST1H3D | Homo sapiens histone cluster 1, H3d (HIST1H3D), mRNA [NM_003530] | NM_003530 |
| ID2 | Homo sapiens inhibitor of DNA binding 2, dominant negative helix-loop-helix | NM_002166 |
| | protein (ID2), mRNA [NM_002166] | |
| IKBIP | Homo sapiens IKBKB interacting protein (IKBIP), transcript variant 1, mRNA | NM_153687///NM_201612 |
| | [NM_153687]///Homo sapiens IKBKB interacting protein (IKBIP), transcript | |
| | variant 2, mRNA [NM_201612] | |
| ITPKC | Homo sapiens inositol-trisphosphate 3-kinase C (ITPKC), mRNA | NM_025194 |
| | [NM_025194] | |
| ITPR1 | Homo sapiens inositol 1,4,5-trisphosphate receptor, type 1 (ITPR1), transcript | NM_002222 |
| | variant 2, mRNA [NM_002222] | |
| LCE1E | Homo sapiens late cornified envelope 1E (LCE1E), mRNA [NM_178353] | NM_178353 |
| LRRFIP2 | Homo sapiens leucine rich repeat (in FLII) interacting protein 2 (LRRFIP2), | NM_006309///NM_017724 |
| | transcript variant 1, mRNA [NM_006309]///Homo sapiens leucine rich repeat | |
| | (in FLII) interacting protein 2 (LRRFIP2), transcript variant 2, mRNA | |
| | [NM_017724] | |
| MDM2 | Homo sapiens Mdm2 p53 binding protein homolog (mouse) (MDM2), | NM_002392 |
| | transcript variant MDM2, mRNA [NM_002392] | |
| MEX3B | Homo sapiens mex-3 homolog B (C. elegans) (MEX3B), mRNA [NM_032246] | NM_032246 |
| NLRX1 | Homo sapiens NLR family member X1 (NLRX1), transcript variant 2, mRNA | NM_170722 |
| | [NM_170722] | |
| PCDH8 | Homo sapiens protocadherin 8 (PCDH8), transcript variant 1, mRNA | NM_002590 |
| | [NM_002590] | |
| PHLDA3 | Homo sapiens pleckstrin homology-like domain, family A, member 3 | NM_012396 |
| | (PHLDA3), mRNA [NM_012396] | |
| PLK3 | Homo sapiens polo-like kinase 3 (PLK3), mRNA [NM_004073] | NM_004073 |
| PPM1D | Homo sapiens protein phosphatase, Mg2+/Mn2+ dependent, 1D (PPM1D), | NM_003620 |
| | mRNA [NM_003620] | |
| PRKAB1 | Homo sapiens protein kinase, AMP-activated, beta 1 non-catalytic subunit | NM_006253 |
| | (PRKAB1), mRNA [NM_006253] | |
| PRKAB2 | Homo sapiens protein kinase, AMP-activated, beta 2 non-catalytic subunit | NM_005399 |
| | (PRKAB2), mRNA [NM_005399] | |
| PTGER4 | Homo sapiens prostaglandin E receptor 4 (subtype EP4) (PTGER4), mRNA | NM_000958 |
| | [NM_000958] | |
| RAPGEF2 | Homo sapiens Rap guanine nucleotide exchange factor (GEF) 2 (RAPGEF2), | NM_014247 |
| | mRNA [NM_014247] | |
| RBM12B | Homo sapiens RNA binding motif protein 12B (RBM12B), mRNA | NM_203390 |
| | [NM_203390] | |
| RPS27L | Homo sapiens ribosomal protein S27-like (RPS27L), mRNA [NM_015920] | NM_015920 |
| RRM2B | Homo sapiens ribonucleotide reductase M2 B (TP53 inducible) (RRM2B), | NM_015713 |
| | transcript variant 1, mRNA [NM_015713] | |
| SEL1L | Homo sapiens TSA305 mRNA, complete cds. [AB020335]///Homo sapiens | AB020335///NM_005065 |
| | sel-1 suppressor of lin-12-like (C. elegans) (SEL1L), mRNA [NM_005065] | |
| SEMG2 | Homo sapiens semenogelin II (SEMG2), mRNA [NM_003008] | NM_003008 |
| SERTAD1 | Homo sapiens SERTA domain containing 1 (SERTAD1), mRNA [NM_013376] | NM_013376 |
| SMAD5 | Homo sapiens SMAD family member 5 (SMAD5), transcript variant 2, mRNA | NM_001001419 |
| | [NM_001001419] | |
| TM7SF3 | Homo sapiens transmembrane 7 superfamily member 3 (TM7SF3), mRNA | NM_016551 |
| | [NM_016551] | |
| TNFRSF17 | Homo sapiens tumor necrosis factor receptor superfamily, member 17 | NM_001192 |
| | (TNFRSF17), mRNA [NM_001192] | |
| TOPORS | Homo sapiens topoisomerase I binding, arginine/serine-rich, E3 ubiquitin | NM_005802 |
| | protein ligase (TOPORS), transcript variant 1, mRNA [NM_005802] | |
| TP53I3 | Homo sapiens tumor protein p53 inducible protein 3 (TP53I3), transcript | NM_004881 |
| | variant 1, mRNA [NM_004881] | |
| TRIAP1 | Homo sapiens TP53 regulated inhibitor of apoptosis 1 (TRIAP1), mRNA | NM_016399 |
| | [NM_016399] | |
| TRIM22 | Homo sapiens tripartite motif containing 22 (TRIM22), transcript variant 1, | NM_006074 |
| | mRNA [NM_006074] | |
| USP41 | ubiquitin specific peptidase 41 [Source: HGNC Symbol; Acc: 20070] | XM_937988 |
| | [ENST00000454608] | |

The TGx-DDI transcriptomic biomarker/expression profile is an established biomarker for the classification of genotoxic and non-genotoxic chemicals. The 64-gene expression profile of TGx-DDI was generated by testing 28 model chemicals (13 that cause DNA damage in cells and 15 that do not cause DNA damage in cells) in human TK6 cells to construct an mRNA gene signature that discriminates between genotoxic and non-genotoxic agents. See, for example, Li et al., Environ. Mol. Mutagen. 56(6): 505-519 (2015); Yauk et al., “Application of the TGx-DDI Transcriptomic Biomarker to Classify Genotoxic and Non-Genotoxic Chemicals in Human TK6 Cells in the Presence of Rat Liver S9,” Environ. Mol. Mutagen. 57: 243-260 (2016); and Jackson et al., “The TGx-DDI biomarker online application for analysis of transcriptomic data to identify DNA-damage-inducing chemicals in human cell cultures,” Environ. Mol. Mutagen. 58:529-535 (2017), all of which are incorporated herein by reference in their entireties. The microarray data for the 28-chemical×64-gene signature profile can be readily accessed at the National Center for Biotechnology Information Gene Expression Omnibus under GEO Series Accession Number GSE58431 (https://www.ncbi.nlm.nih.gov/geo/query/acc.cgi?acc=GSE58431). In addition to the microarray data, as described in the Examples, digital counting was used to obtain a 45 chemical×64 gene signature profile. This profile can be readily accessed at the National Center for Biotechnology Information Gene Expression Omnibus under GEO Series Accession Number GSE107162. (https://www.ncbi.nlm.nih.gov/geo/query/acc.cgi?acc=GSE107162) By comparing mRNA expression data for a test sample, for example, in vitro microarray data, PCR data or digital counting data from a cell contacted with a test agent, to the 28-chemical×64-gene signature profile, or the 45 chemical×64 gene signature profile, the probability that the data fit the profile for a DDI or a NDDI agent can be calculated. As described in the Examples, the data obtained using digital counting highly correlated with the microarray data for the TGx-DDI transcriptomic biomarker. Therefore, one of skill in the art can readily compare a gene expression profile comprising gene expression levels from at least 63 of the genes (e.g., 64 genes) in Table 1, obtained from a cell contacted with a test agent, with the TGx-DDI transcriptomic biomarker/expression profile, to determine whether a test agent is a DDI or a NDDI agent.

As used throughout, gene expression refers to levels of expression, absolute or relative, and/or pattern of expression of a gene. The expression of a gene may be measured at the level of DNA, cDNA, RNA, mRNA, protein or combinations thereof. As used throughout, gene expression profile refers to the levels of expression of multiple different genes measured for the same sample. An expression profile can be obtained from a cell, a cell lysate, or gene products isolated from a cell. in some examples, the expression profile is obtained from a cell contacted with a test agent or a cell lysate of a. cell contacted with a test agent. In other examples, an expression profile is obtained from nucleic acids, for example, mRNA, isolated from a cell contacted with a test agent. In some examples, an expression profile is obtained from a cell, cell lysate or gene products isolated from a cell not contacted with a test agent. The expression profile of a cell not contacted with a test agent can be used as a control, for example, for normalization of expression data, or for comparison with an expression profile of a cell contacted with a test agent.

As used herein detecting or determining expression levels refers to detecting or determining the quantity or presence of an RNA transcript or its expression product. Methods for detecting expression of one or more of the genes set forth in Table 1, that is, gene expression profiling, include methods based on hybridization analysis of polynucleotides, methods based on sequencing of polynucleotides, immunohistochemistry methods, and proteomics-based methods. For example, the amount of an mRNA in a cell can be determined by methods standard in the art for quantifying a nucleic acid in a cell, such as in situ hybridization, quantitative PCR, RT-PCR, Taqman assay, Northern blotting, ELISPOT, microarray analysis, dot blotting, etc., as well as any other method now known or later developed for quantifying the amount of a nucleic acid in a cell. The amount of a protein or a fragment thereof in a cell, can be determined by methods standard in the art for quantifying proteins in a cell, such as densitometry, absorbance assays, fluorometric assays, Western blotting, ELISA, ELISPOT, immunoprecipitation, immunofluorescence (e.g., FACS), immunohistochemistry, etc., as well as any other method now known or later developed for quantifying specific protein in or produced by a cell.

In particular, a global transcriptomic response can be determined using direct digital counting. In these methods, the expression levels of at least 63 genes (e.g. 64 genes) are determined by contacting the isolated RNA with at least 63 probes (e.g. 64 probes), wherein each probe comprises a targeting sequence and a unique molecular barcode sequence and wherein the targeting domain specifically binds to a nucleotide sequence in the mRNA expressed from one of the biomarkers; allowing the probes to hybridize to the mRNAs in the cell lysate; and directly counting the number of unique molecular barcode sequences in the probe/mRNA complexes to determine the expression levels of the genes. Optionally, in any of the methods described herein, two or more probes can be used to detect expression levels of one or more genes in the biomarker. Direct digital counting of nucleic acids can be performed, for example, by using nCounter® technology (Nanostring, Seattle, Wash.), as described in the Examples.

The nCounter® Analysis System uses a novel digital technology that is based on direct multiplexed measurement of nucleic acids and offers unparalleled levels of precision coupled with the ability to quantify up to 800 targets (mRNA, miRNA, or dsDNA) in a single reaction. The nCounter Analysis System is an integrated system comprised of a fully automated prep station, a digital analyzer, the CodeSet (barcodes) and all of the reagents and consumables needed to perform the analysis. See, U.S. Pat. Nos. 7,473,767; 7,919,237; 7,941,279; International Application Nos. WO03/003810; WO08/124847; WO11/100541; WO11/116088; U.S. Pat. Pub. Nos. 2011/0003715; 2011/0207623; 2010/0207623; 2010/0015607; 2010/0261026; 2010/0112710; 2011/0086774; 2011/0201515, which are incorporated herein by reference in their entireties.

In the methods provided herein, digital counting can be used with other methods, for example, PCR-based methods, such as reverse transcription PCR (RT-PCR) and array-based methods such as microarray to obtain expression profiles. The results of expression profiles obtained by one or more of these methods can be combined to identify a test agent as a DDI agent or a NDDI agent, as described herein.

In some examples, the expression detection methods use isolated RNA. The starting material is typically total RNA isolated from a biological sample, for example, a cell or a population of cells. General methods for RNA extraction are well known in the art. In particular, RNA isolation can be performed using a purification kit, a buffer set and protease from commercial manufacturers, such as Qiagen (Valencia, Calif.), according to the manufacturer's instructions. Isolated RNA can be used in hybridization or amplification assays that include, but are not limited to, digital counting, PCR analyses and probe arrays. One method for the detection of RNA levels involves contacting the isolated RNA with one or more nucleic acid molecules (probes) that can hybridize to the mRNA encoded by the gene being detected. The nucleic acid probe can be, for example, an oligonucleotide of between about 5 and 500 nucleotides in length that specifically hybridizes to an mRNA. Hybridization of an mRNA with the probe indicates that the gene in question is being expressed. Optionally the mRNA is immobilized on a solid surface and contacted with a probe, for example by running the isolated mRNA on an agarose gel and transferring the mRNA from the gel to a membrane, such as nitrocellulose. Optionally, the probes are immobilized on a solid surface and the mRNA is contacted with the probes, for example, in an Agilent gene chip array. A skilled artisan can readily adapt known mRNA detection methods for use in detecting the level of expression of the intrinsic genes of the present invention.

In some examples, microarrays are used for expression profiling. By microarray is intended an ordered arrangement of hybridizable array elements, such as, for example, polynucleotide probes, on a substrate. The term probe refers to any molecule that is capable of selectively binding to a specifically intended target biomolecule, for example, a nucleotide transcript or a protein encoded by or corresponding to a gene in Table 1. Probes can be synthesized by one of skill in the art, or derived from appropriate biological preparations. Probes are optionally labeled. Examples of molecules that can be utilized as probes include, but are not limited to, RNA, DNA, proteins, antibodies, and organic molecules.

Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. Microarray analysis can be performed by commercially available equipment, following manufacturer's protocols. such as by using the Affymetrix GenChip technology, or Agilent ink-jet microarray technology.

in the methods described herein, the cell can be any cell suitable for in vitro genetic testing. These include, but are not limited to eukaryotic cells such as, for example, lymphoma cells, lymphoblasts or Chinese hamster cells. Non-limiting examples of cells that can be used in the methods provided herein include L5178Y TK^{+/-} 3.7.2C, TK6, CHO-WBL and CHL/IU cells. In some examples, the cell is a p53 competent cell. By way of example, transcriptomic perturbations in the human lymphoblastoid-derived TK6 cell line can be used. TK6 cells are p53 proficient, well characterized, and extensively used in genotoxicity testing (See Examples and Li et al., 2015). They are robustly responsive in stress signaling studies. In some examples, the cell is contacted with the test agent in the presence of S9 rat liver extract. The cells can be cultured with S9 rat liver extract prior to contacting the cells with the test agent or after contacting the cell with the test agent. In some examples, the methods provided herein include contacting populations of cells with a test agent. In some examples, a subpopulation of cells is isolated from a population of cells prior to contacting the subpopulation of cells with a test agent.

Any of the methods provided herein can be performed as a high-throughput method where a plurality of test agents are screened for genotoxic activity and identified as a DDI agent or a NDDI agent. For example, the methods can comprise identifying about 10, 100, 1000, 10000, 100,000 or more test agents as DDI agents or NDDI agents. To this end, multiple tubes or multiwell plates, including microtiter plates, can be used to screen test agents. For example, 6-, 12-, 24-, 48-, 96-, 384- or 1536-microtiter plates can be used for high throughput assays comprising any of the methods provided herein.

In the methods provided herein the gene expression profile obtained from a cell contacted with a test agent is compared to a gene expression profile for each of a plurality of training samples that have been classified in a class of DDI agents or a class of non-DDI agents, wherein the gene expression profile for each of the plurality of training samples is based on expression of 64 biomarkers from Table 1, and wherein a supervised algorithm was used to construct centroids for each of the DDI and NDDI agent classes in the training set. The centroids for each of the DDI and NDDi agent classes can be constructed using the methods standard in the art, for example, those provided in Li et al., 2015.

As used herein, a supervised algorithm is an approach where a plurality of samples with known subtype or outcome, for example, a DDI subtype or a NDDI subtype, is used to produce a mathematical model that is then evaluated with independent validation data sets. In this case, a training set of gene expression data is used to construct a statistical model that correctly predicts whether a test agent is a DDI agent or a NDDI agent. This training set is then tested with independent data (referred to as a test or validation set) to determine the robustness of the computer-based model. Supervised methods can use a data set with reduced dimensionality (for example, the first few principal components), but typically use unreduced data, with all dimensionality. In all cases the methods allow the quantitative description of the multivariate boundaries that characterize and separate each class in terms of its gene expression profile. It is also possible to obtain confidence limits on any predictions, for example, a level of probability to be placed on the goodness of fit. The robustness of the predictive models can also be checked using cross-validation, by leaving out selected samples from the analysis.

In the methods provided herein, the prediction algorithm can be the nearest shrunken centroids method described in Tibshirani et al., “Diagnosis of multiple cancer types by shrunken centroids of gene expression,” PNAS USA 99: 6567-6572 (2002), which is herein incorporated by reference in its entirety. Briefly, the standardized centroid for each class in a training set is computed, where the standardized centroid is the mean expression level for each gene in each class (or subtype) divided by the within-class standard deviation for that gene. The standard centroid for each class is shrunken toward the overall centroid to produce the nearest shrunken centroid. The method employs a shrinkage parameter to control the number of features used to construct the classifier.

Once the gene expression profile obtained from a cell contacted with a test agent is compared to a gene expression profile for each of a plurality of training samples that have been classified in a subtype of DDI agents or a subtype of non-DDI agents, the distance of the gene expression profile obtained from a cell contacted with a test agent to each of the centroids is calculated to identify the test agent as a DDI agent or a NDDI agent based upon the nearest centroid. It is understood that identification of a test agent as a DDI agent or a NDDI agent is equivalent to assignment of a test agent in the subtype of DDI agents or the subtype of NDDI agents. Methods of calculating the distance of the gene expression profile to each of the centroids are known to those of skill in the art and include, but are not limited to principal component analysis (PCA) (Ma et al., “Principal component analysis based methods in bioinformatics studies,” Brief Bioinform. 12(6): 714-22 (2011)), probability analysis (PA) (Tibshirani et al., 2002), or two-dimensional hierarchical clustering (2DC) (Sturn et al., “Genesis: cluster analysis of microarray data,” Bioinformatics 18(1): 207-8 (2002). As described in the Examples, one or more methods selected from the group consisting of PCA, PA or 2DC can be used to identify the test agent as a DDI agent or a NDDI agent.

Optionally, the data obtained from the gene expression profiles for the training samples and the gene expression profile from the cells contacted with the test agent are processed via normalization methods prior to analysis. In some examples, processing comprises normalization to a set of housekeeping genes. Examples of housekeeping genes include, but are not limited to, G6PD, GUSB, HPRT1, LDHA, NONO, PGK1, PPIH, and TFRC.

Any of the methods provided herein can performed in combination with one or more additional in vitro or in vivo genotoxicity assays, for example, the Ames test (Chaudhary et al. “Evaluation of genotoxicity of Trois through Ames and in vitro chromosomal aberration tests,” Asian Pac. J. Trop Biomed. 3(11): 902-906 (2013)), the in vivo micronucleus test (Hayashi “The micronucleus test-most widely used in vivo genotoxicity test-” Genes Environ. 38: 18 (2016), the in vitro chromosomal aberration test (Ishidate et al. “Chromosome aberration assays in genetic toxicology testing in vitro,” Mutat. Res. 404(1-2): 167-172 (1998)), a comet assay (Speit et al. “The comet assay: a sensitive genotoxicity test for the detection of DNA damage and repair,” Methods Mol. Biol. 314: 275-86 (2006)), or a mouse lymphoma assay (Lloyd et al., “The mouse lymphoma assay,” Methods Mol. Biol. 817: 35-54 (2012)). In vivo assays involving the measurement of the size of a tumor mass in animal models when exposed to drugs can also be combined with any of the in vitro methods described herein to identify a test agent as a DDI agent or a NDDI agent. Any of the methods described herein can be performed, prior to, simultaneously with or subsequent to other in vitro or in vivo testing.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

The term comprising and variations thereof as used herein is used synonymously with the term including and variations thereof and are open, non-limiting terms. Although the terms comprising and including have been used herein to describe various embodiments, the terms consisting essentially of and consisting of can be used in place of comprising and including to provide for more specific embodiments of the invention and are also disclosed.

Publications cited herein and the material for which they are cited are hereby specifically incorporated by reference in their entireties.

A number of embodiments have been described. Nevertheless, it will be understood that various modifications can be made. Accordingly, other embodiments are within the scope of the following claims.

The examples below are intended to further illustrate certain aspects of the methods and compositions described herein, and are not intended to limit the scope of the claims.

### EXAMPLES

The inventory of chemicals mandated by the Toxic Substances Control Act contains 73,757 chemicals that have been reported by manufacturers as being in commercial use as of February 2001, and this number is continually increasing. This poses a serious challenge for regulatory agencies around the world that require thorough assessment of the health effects of chemicals present in the environment and marketplace. In vitro high-throughput screening (HTS) has been proposed as a first-tier screen in chemical assessments. A TGx-DDI high-throughput cell-based assay using the nCounter® system and transcriptomics biomarker, TGx-DDI, meets these needs. The robustness of the TGx-DDI nCounter assay in identifying DDI agents and the concordance with the output of the microarray approach are described herein.

### Materials and Methods

Cell culture and treatment: TK6 cells, a spontaneously-transformed human lymphoblastoid cell line, were grown and treated with chemical agents as described previously (Li et al. (2015) Environ Mol Mutagen 56(6):505-519.). Briefly, exponentially growing cells were treated with the indicated chemical agent for 4 h over a broad dose range, cells were harvested, and total RNA was isolated. RT-PCR was carried out with representative stress genes, which have been shown to be induced by a broad range of stress agents. For agents requiring metabolic activation, treatment of TK6 cells included S9 rat liver extract as described previously (Buick et al., “Integration of metabolic activation with a predictive toxicogenomics signature to classify genotoxic versus nongenotoxic chemicals in human TK6 cells,” Environ Mol Mutagen. 56: 520-534 (2015)). For cell viability assays, at the end of 4 hr treatment, medium was removed from cells, and cells were washed and recovered in fresh medium for 20 hr. Cell viability was measured at the end of recovery period using an MTT Assay kit (Cayman Chemical, Ann Arbor, Mich.).

Microarray procedures: RNA samples from the concentration-setting experiments of each compound at their selected concentrations were pooled together and analyzed using human whole genome expression long-nucleotide probe microarrays (60 nucleotide long, Agilent Technologies) microarray (Li et al., 2015). For consistency with previous results, 2-color microarrays were used, but comparable results have been obtained with single-color microarrays. Each experiment was run on two arrays, and on each array both treated and reference (vehicle control) samples were hybridized in a dye-swap design. Specifically, the reference and treatment samples were labeled with two different fluorescence dyes, Cy3 and Cy5, and then both samples were hybridized onto one array. To reduce the effects associated with different labeling efficiencies, a two-color dye-swapping configuration was used. The results from these two arrays were combined for statistical analysis.

Bioinformatics analyses: Gene expression data were exported from GeneSpring based on Entrez Gene identifiers. Posterior probabilities analysis (PA) for test samples were calculated given the classifier as described in Tibshirani et al. (2002) Proc. Natl. Acad. Sci. 99(10):6567-6572, and implemented in the pamr package for R. Two-dimensional hierarchical clustering (2DC) was conducted using Euclidean distances with average linkage by Genesis (Genesis@genome.tugraz.at). The DDI and NDDI agents from the original training set separated in two main clusters. A chemical clustering with the DDI branch was called DDI, and vice versa for non-DDI agents. Principle component analysis (PCA) was performed using the prcomp function (Venables W N, Ripley B D (2002) Modern applied statistics with S (Springer, New York) in R Bioconductor.

TGx-DDI nCounter Assay: The nCounter™ assay was performed on 100 ng of RNA that had previously been pooled and used in microarray analysis. Details can be found in Geiss et al. “Direct multiplexed measurement of gene expression with color-coded probe pairs,” Nat Biotechnol 26(3):317-325 (2008). In brief, optimized sequences for genes in the TGx-DDI panel were custom-designed and manufactured by NanoString. The CodeSet includes the TGx-DDI gene-set and eight housekeeping genes. Housekeeping genes were selected based on stability and detectable expression levels, including G6PD, GUSB, HPRT1, LDHA, NONO, PGK1, PPIH, and TFRC. The protocol followed standard nCounter instructions (http://www.nanostring.com/media/pdf/MAN_nCounter_Gene_Expression_Assay.pdf) (Geiss et al. Nat Biotechnol 26(3):317-325 (2008). Barcodes were counted for each target and the data were exported. The counts of each target were analyzed using nSolver Analysis software (v3.0) for quality control (QC) and normalization. Normalized data were subjected to further analysis. For developing a high-throughput assay, 5×10⁴ cells/well were seeded in a 96-well plate the day before the treatment. Cells were treated with bleomycin and its corresponding vehicle control, H₂O, for 4 hours, cells were rinsed to remove drug, and then, cells were either lysed in RNA lysis buffer (products from Qiagen (Hilden, Germany), Ambion (Foster City, Calif.), and Promega (Madison, Wis.) were tested and performed comparably) at different concentrations or pelleted for RNA isolation. This treatment was performed in triplicate, and bioinformatics analyses were performed as described above.

### Results

Reproducibility assessment:To demonstrate the technical robustness and reproducibility of the cell culture and exposure conditions, the microarray method, and overall comparability with the learning set data used for TGx-DDI identification, four independent replicate transcriptomic experiments, in which TK6 cells were exposed to 80 μg/ml cisplatin alongside concurrent 0.9% NaCl (vehicle) controls, were conducted. As shown in Table 2, the correlation coefficients across the replicates were above 0.95, indicating that this technical system is highly reproducible.

TABLE 2
Correlation coefficient for cisplatin treatments. Correlation
coefficiency is calculated across the 4 replicate treatments
with 80 μM cisplatin on 432 significantly perturbed genes
relative to solvent controls (p < 0.01 Bonferroni correction).

| Correlation coefficiency | Cisplatin 1 | Cisplatin 2 | Cisplatin 3 | Cisplatin 4 |
|---|---|---|---|---|
| Cisplatin 1 | — | 0.97 | 0.95 | 0.96 |
| Cisplatin 2 | 0.97 | — | 0.95 | 0.95 |
| Cisplatin 3 | 0.95 | 0.95 | — | 0.96 |
| Cisplatin 4 | 0.96 | 0.95 | 0.96 | — |

Four additional agents were selected from the original training set to confirm reproducibility of DDI prediction using the TGx-DDI biomarker. These included a DNA alkylating agent (MMS), a topoisomerase inhibitor (etoposide), an HDAC inhibitor (oxamflatin), and ionizing radiation (4 Gy). Dose-response studies were conducted using the qRT-PCR indicator gene panel comprised of ATF3, CDKNIA, and GADD45A to determine the particular concentration that triggered a robust response for each chemical agent. The microarray results of the three agents, using the selected concentration, and ionizing radiation (IR) were used to classify these agents using the TGx-DDI biomarker, and the expression profiles for each agent was determined. The treatments clustered with their expected categories by 2DC (2-dimensional clustering) using the TGx-DDI biomarker. For the three DNA-damaging agents, the compounds clustered with the genotoxic agents. For oxamflatin, this agent clustered with the non-DNA-damaging agents. Taken together, these experiments demonstrate that this model system and technology generate robust data that are highly reproducible.

Selection of validation compounds: A strategy was developed to evaluate this biomarker with a set of chemicals that covered five classes of distinct genotoxic mechanisms:
Class 1: DDI agents that interact directly with DNA that should be detected as positive in the in vitro CD assays. This group of agents includes alkylating and cross-linking agents, and serves as a positive control for detection of direct DNA-reactive mechanisms.
Class 2: GDDI agents that interact indirectly with DNA. Topoisomerase inhibitors and intercalators are highly potent indirect genotoxicants. Antimetabolites such as nucleoside analogs cause CD in vitro. Some antimetabolites may show effects only after longer exposures than a 4-h assay, and inclusion of these agents tests the limits of the experimental design.
Class 3: Agents that interact indirectly with DNA via effects on cell cycle, regulation of apoptosis, and through interaction with the mitotic apparatus. This class include aneugens that are microtubule inhibitors, which are non-DDI because they cause aneugenicity through spindle interference; and in vitro CD positive kinase inhibitors that are not relevant genotoxicants in vivo because they are typically positive only at doses that are not physiologically relevant.
Class 4: DNA non-reactive compounds that have a ‘clean’ genotoxicity profile including being negative in in vitro CD assays. This class serves as negative controls for testing the transcriptomic biomarker.
Class 5: Compounds that are known to have irrelevant positive results in in vitro genotoxicity assays. This class includes agents like caffeine, nongenotoxic carcinogens, apoptosis inducers, and other chemicals that have been reported positive in in vitro CD assays but for which the genotoxicity findings are understood as irrelevant.

Based on the above, 45 chemicals (Table 3) were used to populate each class.

TABLE 3

| | Sub- | | |
|---|---|---|---|
| Class | class | Definition | CD |
| 1 | | Genotoxins that interact directly with DNA | pos |
| 2 | | Genotoxins that interact indirectly with DNA | pos |
| | 2A | Topo inhibitors including DNA intercalators | |
| | 2B | Antimetabolites | |
| 3 | | Genotoxins that interact indirectly with DNA | pos |
| | | Effect on cell cycle and mitotic apparatus | |
| | 3A | Antimitotic agents | |
| | 3B | Kinase inhibitors (in vitro pos) | |
| | 3C | Additional compounds | |
| | 3D | Heavy metals | |
| 4 | | Non-DNA reactive chemicals, in vitro neg | neg |
| | 4A | Kinase inhibitors (in vitro neg) | |
| | 4B | Non-genotoxic carcinogens | |
| | 4C | General pathways | |
| | 4D | Others | |
| 5 | | Irrelevant positives | pos |

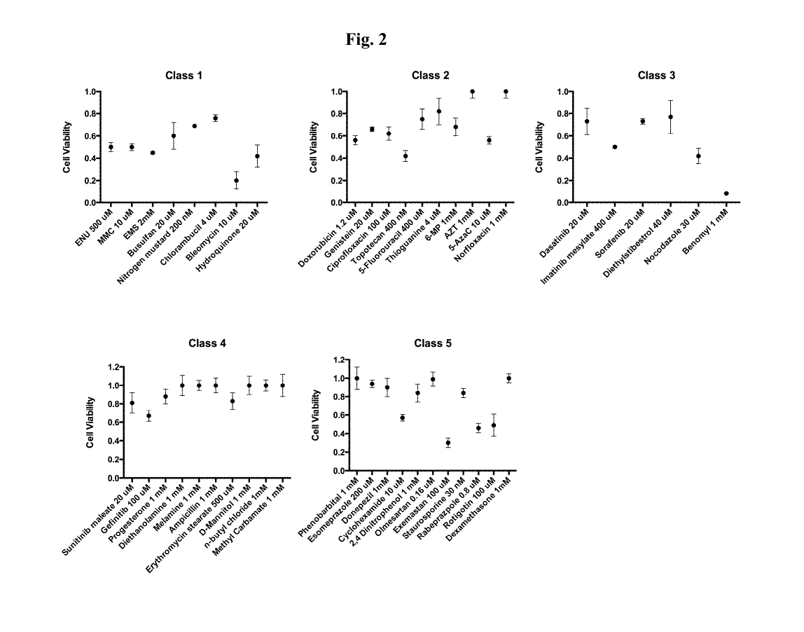

Dose optimization: For some assays, a sufficient concentration of the test agent is required to trigger a measureable transcriptional response; such concentrations may differ from other toxicological endpoints. Therefore, to determine an appropriate concentration for transcriptomics profiling, a dose-range finder experiment was performed for all test compounds as described in Li et al. (2015). Six concentrations of each agent were used for assessment of mRNA changes in three indicator genes (ATF 3, CDKNIA, and GADD45A) by qRT-PCR. The concentration for each agent showing the strongest induction of the indicator genes was then selected. In addition, concordance of responses in the indicator genes was confirmed prior to pooling samples for microarray analysis. If none of the indicator genes were induced in concentration-setting experiments, the IC₅₀ was selected for the microarray analysis. The IC₅₀ value was determined using a standard MTT assay at 24 hr using 10 concentrations and 3 replicates. Based on this cytotoxicity assay, the selected concentrations for microarray analysis were not overtly cytotoxic for any test agent (FIG. 2). Based on this cytotoxicity assay, the selected doses for microarray analysis were not overtly cytotoxic. If there was neither cytotoxicity nor induction of expression changes in the gene panel, a concentration of 1 mM was used for microarray analysis, as per the revised ICHS2(R1) guidance on genotoxicity testing of pharmaceuticals (ICHS2(R1) (2012) Guidance for Industry: S2(R1) Genotoxicity testing and data interpretation for pharmaceuticals intended for human use. US Department of Health and Human Services, Food and Drug Administration http://www.ich.org/fileadmin/Public_Web_Site/ICH_Products/Guidelines/Safety/S2_R1/Step4/S2R1_Step4.pdf). Selection of a single concentration and pooling replicate samples for microarray analysis (described below) is specific to biomarker development and validation, where multiple compounds were used in each class; future application in substance testing should be undertaken using a dose-response design with samples in triplicate (Buick et al., 2017).

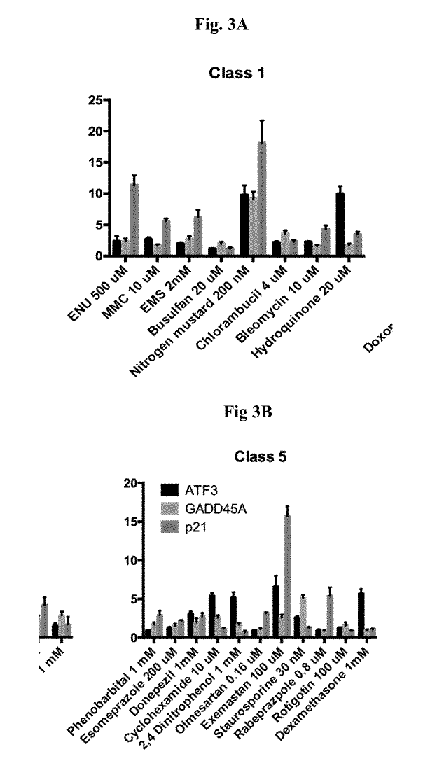

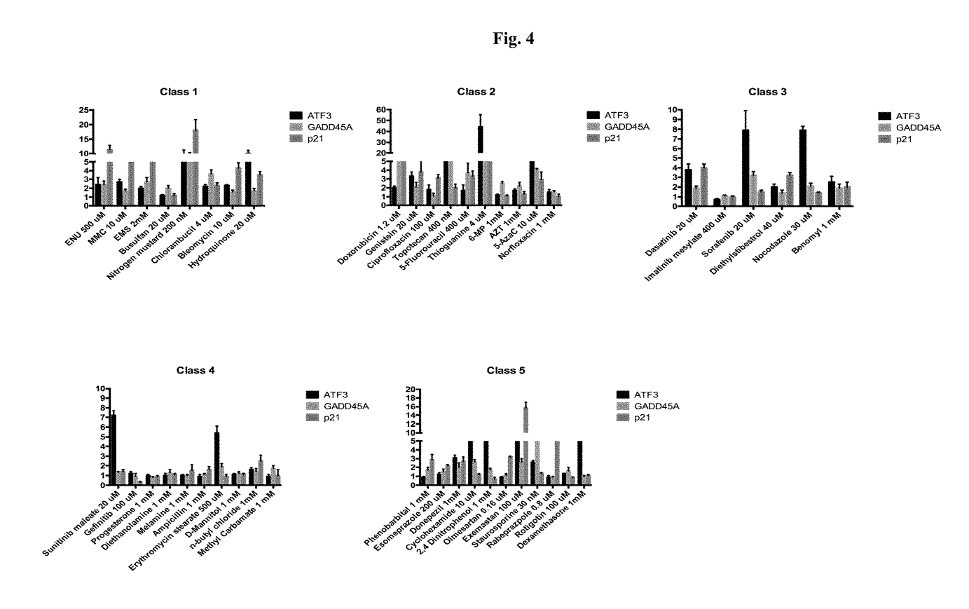

The induction of stress genes at the selected concentration for chemicals in Class 1 and 5 is shown in FIGS. 3A and 3B, respectively. All chemicals except busulfan in Class 1 induced robust responses in at least one gene at the selected concentration. Only GADD45A was induced by at least 2-fold in cells treated with busulfan at the selected concentration. Higher concentrations of busulfan did not cause greater induction of these stress genes suggesting transcriptional inhibition at high concentrations. The treatment of all but one compound (bleomycin) at the selected concentrations resulted in a reduction in cell viability by at least 30% at 24 hr (FIG. 2). However, cells treated with bleomycin showed an 80% decline in viability at 24 hr. The concentration determination of Class 5 compounds was based on qRT-PCR results, except for rotigotin, which did not induce any of the indicator genes at the concentrations tested, including cytotoxic ones. Therefore, the IC₅₀ for cell viability was selected for the microarray experiment. In contrast, the other compounds in Class 5 induced at least one of the three indicator genes at the selected concentrations. Aside from exemastan, rabeprozpole, and rotigotin, the remaining Class 5 compounds were not cytotoxic with viability >80% of control (FIG. 2). The concentration determination results of all five classes can be found in FIG. 4.

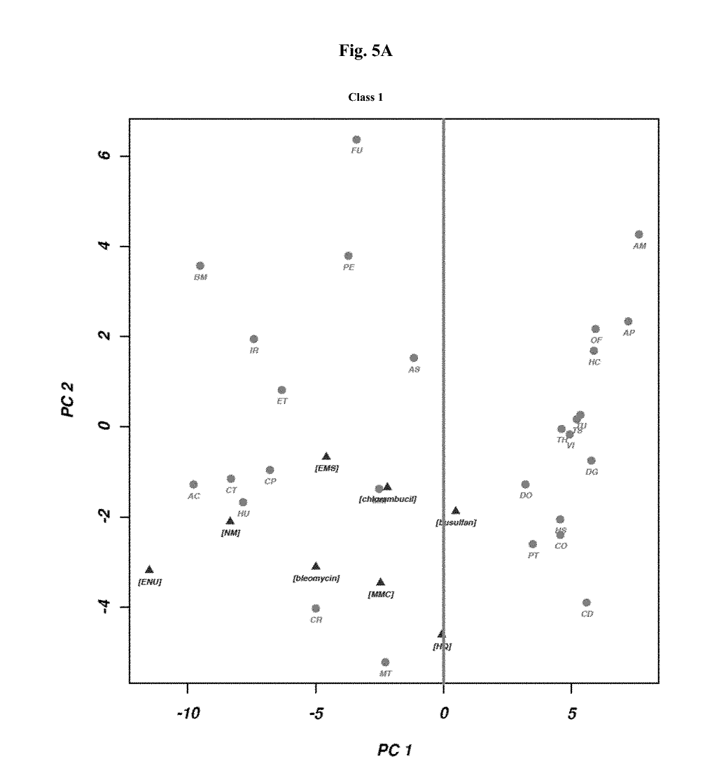

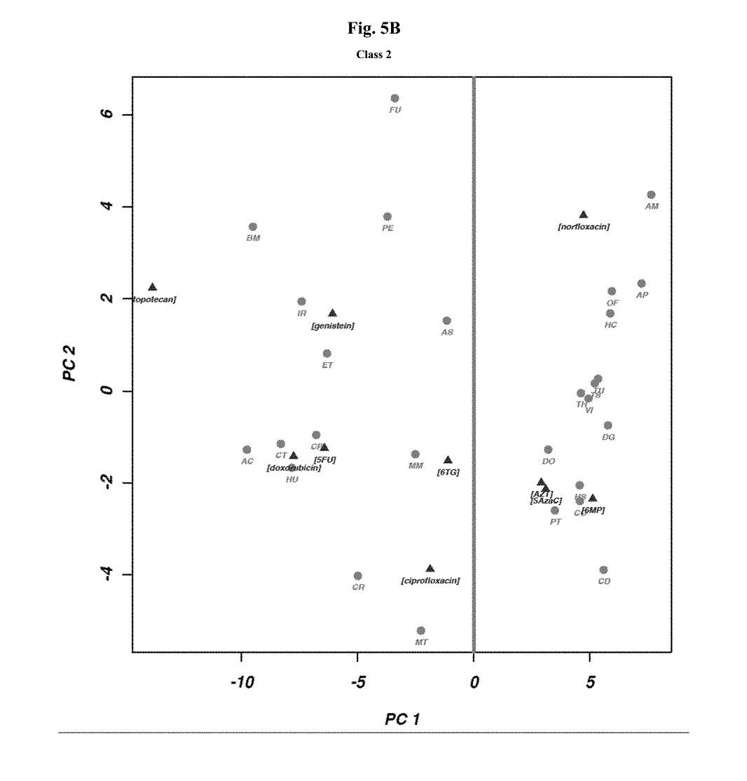

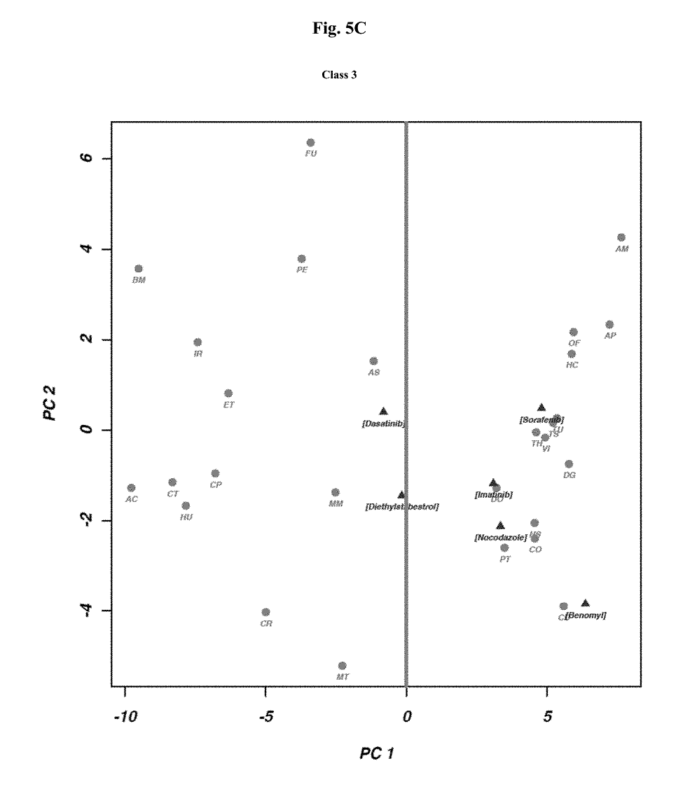

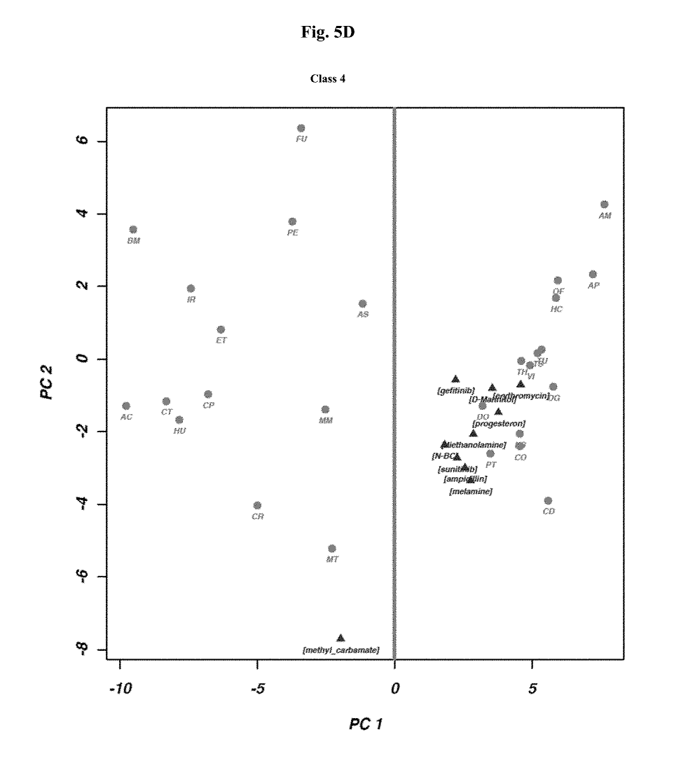

TGx-DDI transcriptomic biomarker evaluation: Following the dose determination, microarray analysis was performed for each test compound. RNAs from three replicates in the concentration-setting experiment were pooled together and used for microarray analysis. Cisplatin and ionizing radiation were used in parallel during each experiment as positive controls and to assess batch variation. The TGx-DDI transcriptomic biomarker panel was used to classify each chemical as DDI or non-DDI using two-dimensional clustering (2DC), principal component analysis (PCA), and probability analysis (PA). Hierarchical clustering and PCA were used as an initial unsupervised method to explore the data. A category assignment was first determined by the position of the test chemical in the tree structure of the dendrogram generated by 2DC, or in the PCA plot (FIG. 5(A)-(E) for Class 1-5, respectively. Finally, TGx-DDI-based DDI prediction was conducted by applying the shrunken centroids approach—posterior probability analysis. This was done by determining distances of gene expression changes for each of the biomarker genes from the DDI and non-DDI centroids. A DDI call was based on p>0.9 of the compound being in that class, and vice versa for a non-DDI call. A chemical was considered ‘unclassified’ if it did not meet these criteria.

The TGx-DDI heatmap for chemicals in all five classes. PA, PCA, and 2DC results using the TGx-DDI biomarker for validation chemicals were generated. In order to decrease the probability of false negatives, a three-pronged approach was used. A chemical was classified as DDI if it gave a positive call in any one of the TGx-DDI biomarker analyses described in the paragraph above (2DC, PCA or PA prediction). A chemical was classified as non-DDI if it did not meet any of these criteria.

Overall, all agents in Class 1 were classified as DDI, all agents with one exception (methyl carbamate) in Class 4 were classified as non-DDI, and all except one agent in Class 5 (exemastan) were classified as non-DDI. Class 3 agents were classified as non-DDI with two exceptions (both dasatanib and diethylstilbestrol gave genotoxic calls). More than half of the Class 2 agents gave DDI calls.

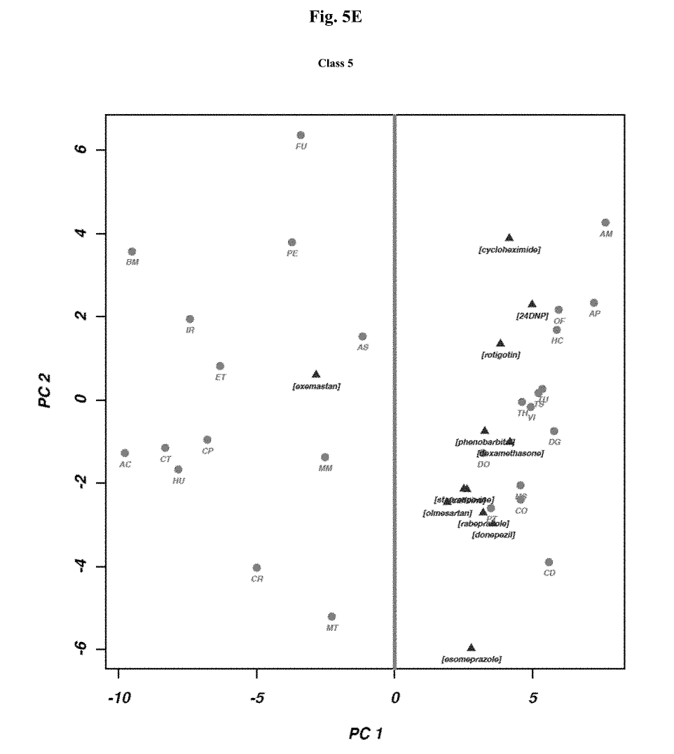

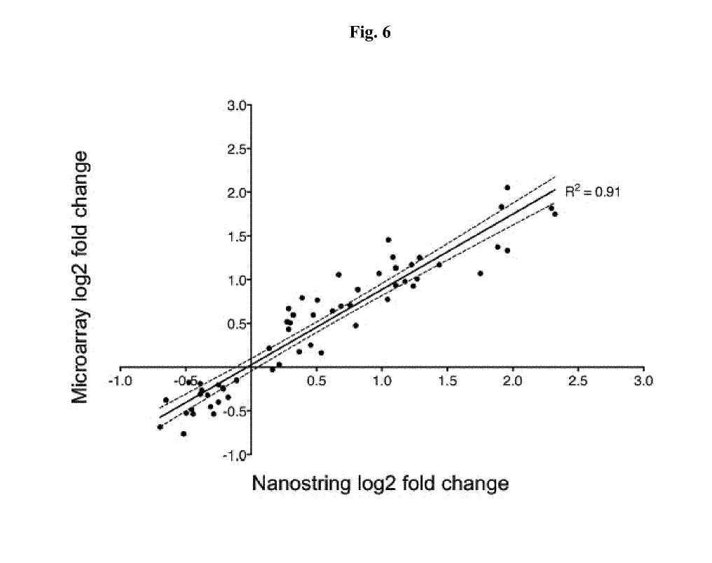

Development of the TGx-DDI nCounter Assay: To meet the need of a multiplex detection system suitable for high-throughput screening (HTS) application measuring a multi-gene transcriptomic biomarker, a TGx-DDI assay was developed applying nCounter, a direct digital counting technology. First, the robustness of TGx-DDI nCounter assay was assessed by comparing the results of the 28 training-set agents in TK6 cells to those using microarrays. The TGx-DDI codeset includes an optimized TGx-DDI gene set and eight housekeeping genes. Housekeeping genes were selected based on stability and detectable expression levels. A high correlation was observed between nCounter assay (FIG. 6) and microarray results for TGx-DDI.

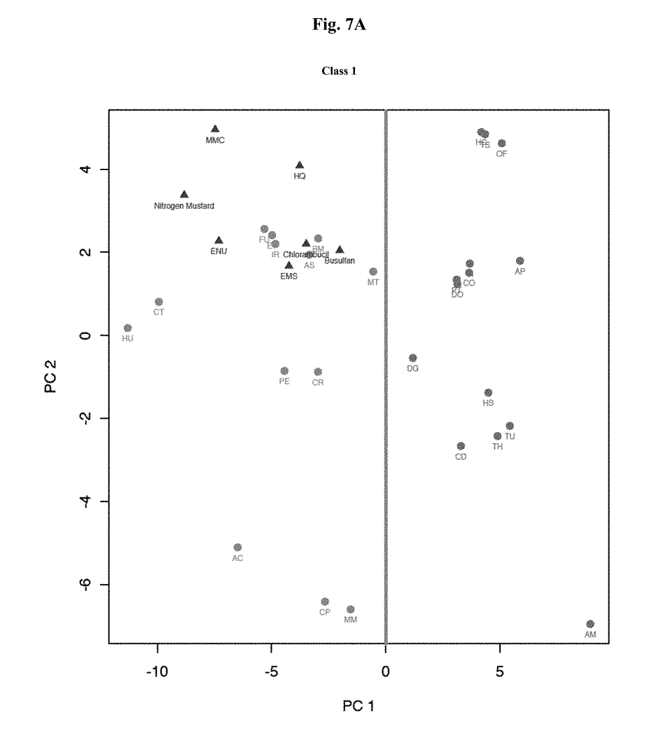

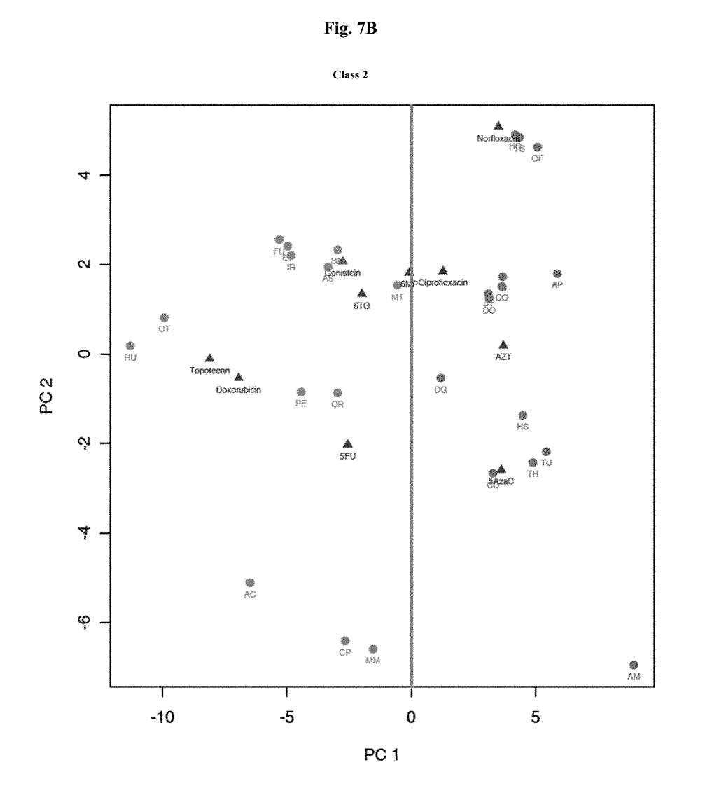

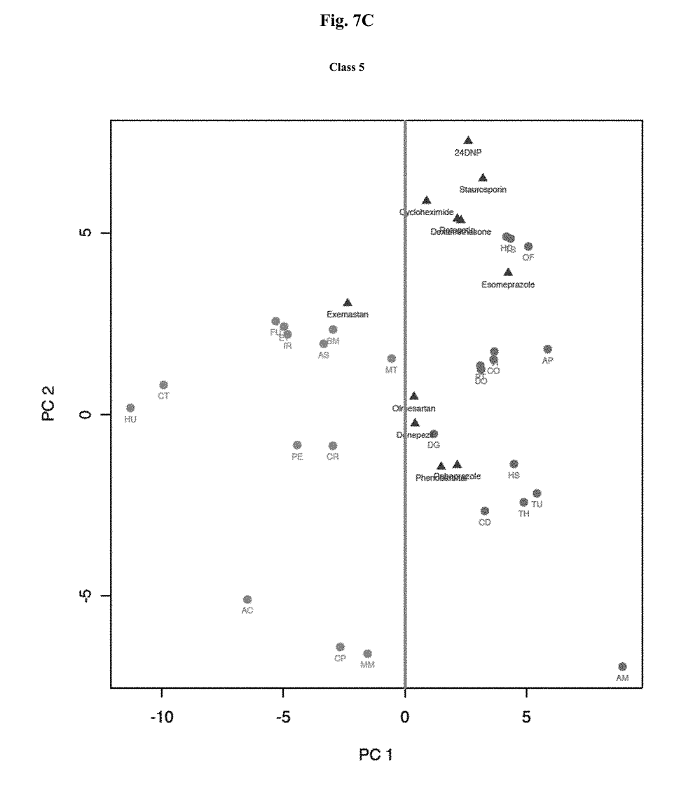

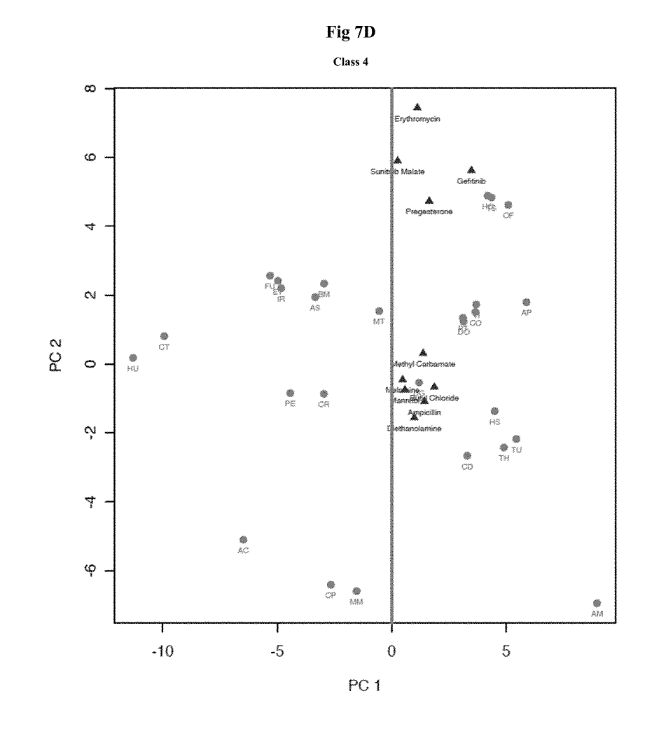

To validate the sensitivity and specificity of DDI prediction by TGx-DDI nCounter assay, 38 out of 45 testing compounds, including Class 1, 2, 4 and 5, were evaluated using nCounter technology. nCounter assays were performed on 100 ng of total RNA, the same RNA samples that were used in microarray analysis. Following the categorization guideline described already for microarrays, compounds were classified as DDI or non-DDI based on nCounter assay data. In addition to the 38 compounds in four classes, additional chemicals requiring metabolic activation were validated. The heatmap of the training set of 28 agents was created using TGx-DDI nCounter assay, and the heatmap for compounds in different classes and compounds requiring metabolic activation was generated. By comparing to microarray results (FIG. 3), the classification of the majority of compounds was consistent between these two platforms; however, responses to several weak DDI compounds were better detected by nCounter. For instance, as mentioned above, both busulfan and hydroquinone were predicted as non-DDI by PA, while 2DC and PCA indicated that these are DDI agents using microarray data. The analysis of the nCounter data for these two agents showed consistency among three classification methods suggesting that the nCounter system is surprisingly more sensitive for detecting responses to weak DDI agents. Moreover, all agents in Class 4 were classified using the nCounter system as non-DDI, which is 100% consistent with CA assay results, while 9 out of 10 agents were classified as non-DDI agents by microarray, summarized in Table 4. The results of 2DC and PCA analyses for TGx-DDI nCounter assay are shown in FIG. 7(A)-(E). The classification of agents in the presence of S9 metabolic activation was also consistent with expectations, demonstrating that the method can be used accurately with S9.

TABLE 4
Consistency of TGx-DDI prediction (by microarray and nCounter
methods) with CA assay results for selected test classes.

| | | Class 2 | | | |
|---|---|---|---|---|---|
| Technology | Class 1 | Class 2A | Class 2B | Class 4 | Class 5 |
| microarray | 100% | 80% | 40% | 90% | 9% |
| | (8/8) | (4/5) | (2/5) | (9/10) | (1/11) |
| nCounter | 100% | 80% | 60% | 100% | 9% |
| | (8/8) | (4/5) | (3/5) | (10/10) | (1/11) |

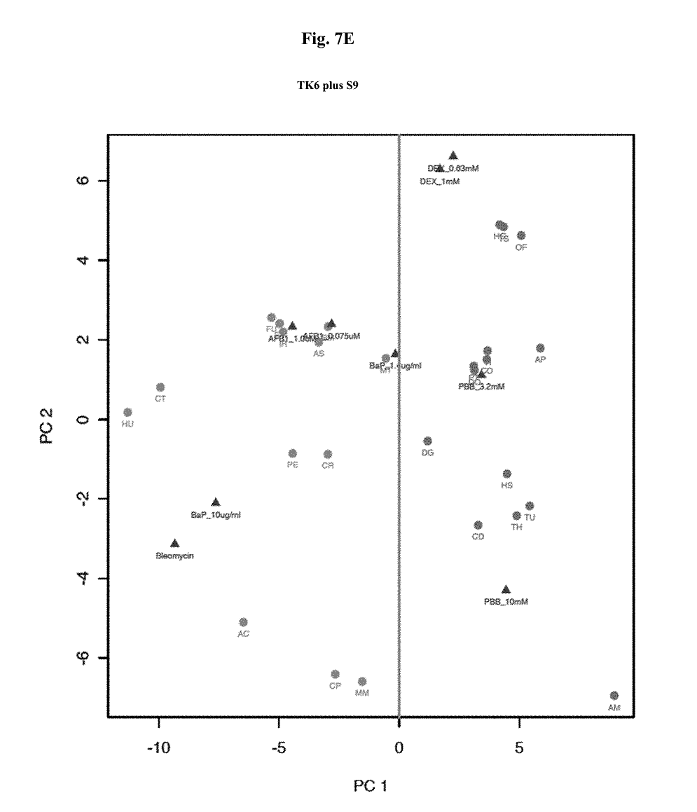

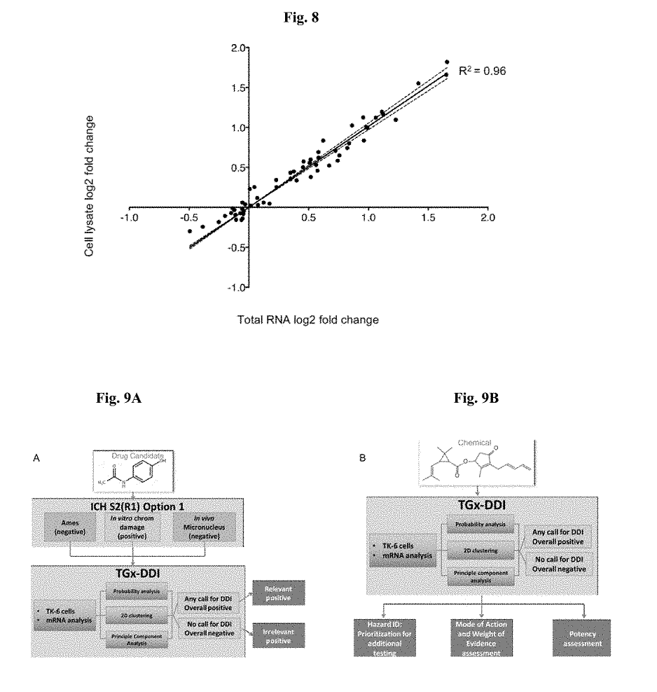

As microarray-based toxicogenomic methods do not support high-throughput, it is time consuming and very costly to perform such assays with multiple chemicals and varying treatment conditions (e.g., only one dose for each chemical was used for microarray analysis). The high-throughput capability of the nCounter system makes multi-condition testing much more feasible. To develop a HTS TGx-DDI nCounter assay, crude cell lysates were tested in addition to isolated RNA with this technology, using solvent- or bleomycin-treated TK6 cells as samples. This method omits RNA extraction steps so that it can be coupled to nCounter measurement for a highly automated HTS system. As shown in FIG. 8, nCounter results of cell lysates at various cell concentrations showed comparable results to that of purified RNA from the original bleomycin and solvent control experiments, and yielded correlation coefficients of 0.90-0.96 in fold changes for the TGx-DDI biomarker genes from pure RNA extracts versus cell lysates.

The nCounter® Analysis System enables the profiling of up to eight hundred of mRNAs or microRNAs simultaneously with high sensitivity and precision. The primary benefits of the platform are the ability to complete studies faster and with very high precision. Faster time to completion of studies is enabled by nCounter®'s streamlined workflow, high sample throughput, and multiplexing capability. nCounter®'s digital counting capability provides highly reproducible data over 5 logs of dynamic range and does not require any amplification steps that might introduce bias to the results. An additional advantage of nCounter® chemistry is that it is highly tolerant of difficult sample types such as formalin-fixed and paraffin-embedded (FFPE) tissue and crude-cell lysates.

The system utilizes a digital technology that is based on direct multiplexed quantification of nucleic acids and offers high levels of precision and sensitivity. Specifically, molecular barcodes and single molecule imaging are employed to detect and count hundreds of unique targets in a single reaction.

NanoString's nCounter® technology is based on direct detection of target molecules using color-coded molecular barcodes, providing a digital count of the number of target molecules. The probe pair consists of a Reporter Probe, which carries the signal on its 5′ end, and a Capture Probe which carries a biotin on the 3′ end (See, for example, Tekletsion et al. “Gene detection and expression profiling of Neisseria meningitides using NantoString nCounter platform,” J. Microbiol. Methods 146: 100-103 (2018); Pascoe et al. “Gene expression analysis in asthma using a targeted multiplex array,” 17(1): 189 (2017), both of which are incorporated herein in their entireties by this reference. The color codes carry six positions and each position can be one of four colors, thus allowing for a large diversity of codes that can be mixed together in a single reaction tube for direct in-solution hybridization to target and yet still be individually resolved and identified during data collection.

Purification and binding of the hybridized complexes is carried out automatically on the nCounter® Prep Station. Magnetic beads, derivatized with short nucleic acid sequences that are complementary to the capture Probe and the reporter probes are used sequentially. First, the hybridization mixture is allowed to bind to the magnetic beads by the capture probe. Wash steps are performed to remove excess reporter probes as well as DNA fragments that are not hybridized. After washing, the capture probes and target/probe complexes are eluted off of the beads and are hybridized to magnetic beads complementary to the reporter probe. Wash steps are performed and excess capture Probes are washed away. Finally, the purified target-probe complexes are eluted off and are immobilized in the cartridge for data collection.

Data collection is carried out in the nCounter® Digital Analyzer. Digital images are processed and the barcode counts are tabulated, for example, in a comma separated value (CSV) format.

With only 4 pipetting steps per sample, thousands of data points are generated with approximately fifteen minutes hands-on time and with fewer errors than conventional methods. The result is a highly reproducible, sensitive assay that provides data spanning a wide dynamic range. Each color-coded barcode represents a unique target molecule. Barcodes hybridize directly to target molecules and can be individually counted without the need for amplification, providing very sensitive digital data. The complete workflow comprises three steps:
1. Hybridization: Two probes hybridize directly to a target molecule in solution. The reporter probe carries the fluorescent barcode and the capture probe contains a biotin moiety that immobilizes the hybridized complex for data collection.
2. Purification and immobilization: After hybridization, samples are transferred to an nCounter® instrument, which removes excess probes. Purified target-probe complexes are bound, immobilized, and aligned on the imaging surface of the nCounter® Cartridge.
3. Count: Sample cartridges are scanned by an automated fluorescence microscope. Barcodes are counted for each target molecule, and the data are exported as a simple CSV file.

The configuration of the analysis system provides the option to expand throughput for analyzing differential gene expression. It starts with one Prep Station and one Digital Analyzer. A second Prep Station can be added on when needed to double capacity. Two nCounter® Prep Stations and a separate Digital Analyzer help eliminate bottlenecks in sample processing and data collection. A single operator can process 96 lanes per day and up to 384 samples with additional sample plexing. For GLP compliance, an enterprise software package is included for laboratories that require enhanced security. Control user access, automate data flow, and generate audit logs.

The nCounter® Prep Station is the automated liquid handling component of the nCounter® Analysis System. It processes samples post-hybridization to prepare them for data collection on the nCounter® Digital Analyzer. Prior to placing samples on the Prep Station, samples are hybridized according to the nCounter protocol. On the deck of the Prep Station, hybridized samples are purified and subsequently immobilized in the sample cartridge for data collection.

The nCounter® Digital Analyzer collects data by taking images of the immobilized fluorescent reporters in the sample cartridge with a CCD camera through a microscope objective lens. Images are processed internally and the data output files include the target identifier and count number along with a comprehensive tally of internal controls that allows each assay to be quantitative. The small data files can be distributed using a variety of methods and are easily integrated with commonly used data analysis and visualization packages.

The use of nSolver™ Analysis Software (Nanostring Technologies, Inc.) allows interrogation of data quickly and efficiently. The unique software with its advanced module further expedites analysis. nSolver™ Analysis software has the ability to quickly and easily QC, normalize, and analyze nCounter® data without the need to purchase additional third-party software packages. nSolver™ is compatible with Windows and Macintosh operating systems, and it can perform the significance testing and calculate the fold changes. In addition, this product integrates with, or exports data in formats compatible with commercially available software packages designed for more sophisticated analyses and visualizations. The recently release v3.0 nSolver™ Analysis Software enables new, advanced analysis modules to perform a wide range of automated data analysis and visualization tasks, and thus reduces personnel time.

Direct, digital counting technology reduces the risk of signal saturation, providing reliable data across a wide dynamic range. Many genes are expressed at less than one transcript per cell and could be measured with less than 15% CV, allowing quantitative measurements of 2-fold changes or less. Precision increases with level of expression, in some cases allowing for quantification of less than 1.2-fold. Correlation between technical replicates often exceeds 0.99. Lot-to-lot and site-to-site variability is also minimal, facilitating long-term studies across multiple independent testing sites. Further, nCounter® output correlates with other transcriptomic platforms (e.g., an ABI StepOnePlus qPCR system and mRNA-seq dataset)

nCounter® assays can accept samples such as purified total RNA, raw cell or blood lysates, and formalin-fixed paraffin-embedded (FFPE) extracts with no loss in precision. Even severely degraded RNA can be a viable sample input. Many nCounter® assays require only 100 ng or less of input material, which would be ideal for applications with cell types where cell number may be limiting. Three cell lysates (2,500, 5,000, and 10,000 cells) were used during sample hybridization and compared to 100 ng of purified total RNA. Results using cell lysates were highly correlated with purified RNA (R2>0.97 for all three) and demonstrated that comparable data can be achieved with either protocol.

The nCounter Analysis System is used to measure gene expression of up to 800 genes in a single assay and identify those genes that change significantly between samples. This approach is different from the global profiling transcriptomic methods such as expression microarrays and RNAseq in that the nCounter system measures an established biomarker comprised of multiple genes. The NanoString nCounter technology is ideal for the late stages in the applying the validated transcriptomic biomarkers, such as the TGx65. The overall performance of the nCounter system correlated well with both microarrays and RNAseq in head-to-head comparisons with the same total RNA samples. In addition, the nCounter system is more sensitive than microarrays because of the in-solution hybridization and amplification-free digital counting method.

The only system comparable to nCounter for multiplex gene expression measurement is the qRT-PCR platform. However, nCounter has advantages over qRT-PCR: First, the sample RNA is measured directly without amplification. Thus, no gene-specific or 3′ biases are introduced, and the levels of each transcript within a sample can be established by counting the number of molecules of each sequence type and calculating concentration with reference to internal standards. In contrast, in real-time PCR, transcript concentration is calculated from the number of enzymatic steps required to attain a threshold level of product. Second, nCounter provides a digital readout of the amount of transcript in a sample. A pure digital readout of transcript counts is linear across a large dynamic range, exhibits less background noise and is less ambiguous for downstream analysis than technologies that use analog signals. Finally, the cost, time, effort and sample requirements of the nCounter system are significantly more efficient than real-time PCR. For example, to measure 500 genes using 2 ng of RNA per real-time PCR reaction in triplicate, one would need 3 mg of total RNA and 1,500 reactions whereas the same experiment could be performed using the nCounter system with 300 ng of total RNA in three reactions. A full time operation for the nCounter Max system only requires ¼ FTE.

### Discussion

The first objective of this study was to provide validation data to support the qualification of the TGx-DDI biomarker for assessing genotoxic hazard and de-risking compounds with isolated in vitro positive chromosome damage findings. The integrated TGx-DDI bioinformatics approach has high accuracy for classification of DDI agents and non-DDI agents and is highly effective in differentiating relevant from irrelevant chromosome damage assay findings. Based on the results of these studies, a standardized workflow was developed for application of the TGx-DDI biomarker in genetic toxicology risk assessment (FIG. 9). As shown in FIG. 9, the TGx-DDI transcriptomic biomarker can be applied during assessment of pharmaceuticals (FIG. 9A) and environmental/industrial chemicals (FIG. 9B). In pharmaceutical assessments with positive results from in vitro mammalian cell chromosome damage assays, the biomarker provides insight into the relevance of these positive findings for agents that are otherwise negative in Ames and in vivo tests (FIG. 9A). This is important because the human relevance of a positive in vitro CD finding still necessitates multiple in vivo follow-up studies despite a negative in vivo genotoxicity test (ICHS2(R1). Thus, the risk assessment of these positive in vitro findings is a challenge to industry and regulatory agencies. The studies described herein demonstrate that the application of the TGx-DDI transcriptomic biomarker will add significant value to the current genotoxicity testing battery for pharmaceuticals by reducing the need for complicated follow up in vitro and in vivo tests and streamlining what animal tests are needed. For industrial and environmental chemicals, the TGx-DDI provides a feasible high throughput approach for detecting and characterizing genotoxicity hazard (FIG. 9B). Specifically, the biomarker could be used in HTS for identifying and prioritizing what agents may cause DNA damage when large chemical sets require assessment. In addition, as in the pharmaceutical application, the biomarker can be used in parallel with conventional in vitro genotoxicity tests to provide weight of evidence in genotoxicity hazard assessment, aid in differentiating DDI from non-DDI (i.e., aneugenicity) modes of genotoxic action, and provide insight into potentially irrelevant positives. Finally, the response of the biomarker genes is useful to determine a chemical's genotoxic potency when run in parallel with prototype agents (Buick et al., 2017).

Overall, this transcriptomic biomarker approach has the potential to complement and/or eventually replace standard genotoxicity assays by providing information about biological responses to genotoxic stress that is not obtained using current methods. While the standard current in vitro genotoxicity assays, particularly CD and MLA, give a phenotypic readout, the TGx-DDI provides insight into molecular responses by a toxicant. Specifically, a positive response using the TGx-DDI biomarker indicates that sufficient DNA damage was incurred and recognized by the cell to initiate a transcriptional DNA damage response that is driven by DNA-damage-response signaling including p53. Moreover, the pattern of transcription induction by p53 differs between genotoxic agents, and these profiles may be useful in classifying mechanisms of action.

This validation study comprised an assessment of 45 test chemicals across five recommended mechanistic classes using a transcriptomics profiling approach. The results of the TGx-DDI toxicogenomics assay and the data from standard genotoxicity testing assays were determined for these 45 test chemicals. The TGx-DDI biomarker was applied using the three statistical approaches (2DC, PCA, and PA). The individual results of each statistical model, as well as the overall call given (positive in any model is a positive call) were obtained. The results of the 2DC, PCA and PA are consistent in general. In more than 90% of the test cases the results from these three analyses agree with each other (i.e., only three out of the 45 agents show differing results). Three agents, namely busulfan, hydroquinone, and diethylstilbestrol, are predicted as non-DDI by PA, but 2DC and/or PCA analysis are positive indicating that these compounds are DDI. These three agents are exceptions because they induce weaker gene expression responses overall than the other positive agents based on visual inspection of the heatmap and are positioned very close to the cutoff line in the PCA plot. Together with the negative PA result, the data show that these agents cause relatively weak genotoxic effects under our test condition. Thus, in order to ensure as few false negatives as possible in compound screening/assessment and thereby keep high sensitivity, agents that induce weak TGx-DDI responses are also reported as DDI if one or more analysis is positive.

The TGx-DDI biomarker classifies all agents in Class 1 as DDI, consistent with results for these compounds using in vitro chromosome damage and Ames assays. In addition, all of the non-DDI agents in Class 4, except for methyl carbamate, are classified as non-DDI when applying the TGx-DDI transcriptomic biomarker analytical approach, again consistent with in vitro chromosome damage and Ames assays. An antibiotic with topoisomerase inhibitory activity, norfloxacin in Class 2A, is predicted to be non-DDI by the TGx-DDI biomarker, while the in vitro chromosome damage and Ames assay results are positive and negative, respectively. It is known that the fluoroquinolone antimicrobials target bacterial DNA gyrase and topoisomerase IV and that the effect on eukaryotic topoisomerase is weak, and the relevance of genotoxicity depends on difference in affinity between the bacterial gyrase and mammalian topoisomerase. Overall the mammalian topoisomerase inhibitors were all identified by the biomarker. In Class 2B, three antimetabolites, 6-mercaptopurine (6-MP), azidothymidine (AZT), and 5-azacytidine (5AzaC), are classified as non-DDI, while the other two anti-metabolites, 5-FU and 6-TG, are predicted to be DDI by TGx-DDI. This difference may reflect the different mechanisms of action. Unlike 5-FU and 6-TG, both of which can incorporate into DNA and block DNA synthesis (i.e., a signal adequately detected by TGx-DDI), AZT and 5AzaC interfere with reverse transcriptase and DNA methylation, respectively. 6-MP affects purine nucleotide synthesis by inhibiting phosphoribosyl pyrophosphate amidotransferase, a rate-limiting enzyme for purine synthesis, which does lead to genotoxicity, but the effects may not be evident until later time points. Indeed, as assessed by qRT-PCR of the indicator genes a stronger transcriptional response to 6-MP and 5AzaC was evident at a later time point. Thus, the biomarker may have some limitations in assessing antimetabolites. However, in most cases the antimetabolite properties of compounds can be easily predicted based on chemical structure. In the case of kinase inhibitors, the biomarker could be triggered by alteration of signaling pathways, which is irrelevant to genotoxic risk. As described above, these cause genotoxicity only at concentrations that are not physiologically relevant. Overall the classifier was effective in differentiating relevant and irrelevant findings. Therefore, our results indicate that application of the biomarker in genotoxicity testing would significantly increase efficiencies in de-risking irrelevant positives in chromosome damage assays.

Even though it is not highlighted in the workflow (FIG. 9), dose selection is one of the key processes for the robustness of the TGx-DDI assay. The aim of the toxicogenomic assay is to assess the toxicity by measuring the stress-responsive transcript signatures, which may not necessarily correlate to cytotoxicity endpoints. The cytotoxicity at the doses selected for these 45 chemicals varies substantially even within the same class. The qRT-PCR dose optimization approach monitors a panel of three well-characterized stress genes, ATF3, CDKN1A, and GADD45A, which serves as an indicator for effective transcriptional response to the treatments. Although TGx-DDI is a robust biomarker and the accurate genotoxicity prediction can be achieved in a range of different doses based on the dose-response study using TGx-DDI(Amundson et al., “Stress-specific signatures: Expression profiling of p53 wild-type and -null human cells,” Oncogene 24: 4572-4579 (2005)) the dose optimization by qRT-PCR is suggested as a standard procedure for this toxicogenomics application. The xenobiotic-induced transcriptional responses can be blunted at very high concentrations at which transcriptional machinery or cell integrity is compromised and the marginal response at low doses can compromise the prediction of toxicity. Thus, dose optimization procedure provides a standardized condition for a tested agent at the selected dose, and decreases the likelihood of false negatives.

The present assay is not limited to the specific array platform or technology described herein, as data collected using other array platforms (e.g., Doktorova et al. (2013) Transcriptomic responses generated by hepatocarcinogens in a battery of liver-based in vitro models. Carcinogenesis 34(6):1393-1402) or with RNA-seq technology (Yauk et al. (2016) Application of the TGx-DDI transcriptomic biomarker to classify genotoxic and non-genotoxic chemicals in human TK6 cells in the presence of rat liver S9. Environ Mol Mutagen) can also be analyzed using the TGx-DDI biomarker. The TGx-DDI classifier can also predict DNA damage in the presence of rat liver S9 in human TK6 cells. Interestingly, the TGx-DDI biomarker was able to predict DDI agents using published Affymetrix array data in HepaRG cells, a metabolically competent human liver hepatocyte cell line. Thus, in addition to confirming the utility of the TGx-DDI biomarker in the presence of S9 and in a different cell line, the work also provides further validation for the TGx-DDI classifier overall by demonstrating its efficacy in an independent data set produced in two separate laboratories using different technologies and its transferability across laboratories. As the biomarker is enriched in p53-responsive genes, the use of p53-competent cells is mandatory for the assay.

Since this biomarker is comprised of less than 100 genes, it is feasible to develop it into the high-throughput screening application. The nCounter Analysis System enables the profiling of up to hundreds of transcripts simultaneously with high sensitivity and precision; therefore it is an ideal system to measure an established biomarker comprised of multiple genes. The results of the TGx-DDI nCounter assay have shown that nCounter is an excellent technology platform for TGx-DDI. First of all, nCounter is equivalent to microarray in terms of de-risking the agents with irrelevant positive CA results by using the TGx-DDI biomarker. Second, the output of nCounter has been found to be more sensitive than microarray. Responses of several weak DDI compounds are better detected by nCounter without compromising the specificity. Third, the high-throughput capability of the nCounter system allows for the development of a highly-automated workflow requiring minimal hands-on time for the large-scaled multi-condition screening. The HTS approach directly using cell lysates also allows for cost-efficient analyses at multiple doses and conditions, in contrast to microarray approaches.

TGx-DDI is the first genotoxicity biomarker that not only shows convincing inter- and intra-laboratory reproducibility but also performs robustly and consistently on different assay platforms. This biomarker can be used in a simple, inexpensive and rapid method which can be easily integrated into the safety evaluation of compounds and chemical series. The incorporation of the genomic biomarker-based genotoxic risk assessment would reduce animal testing. Considering that many chemical agents cannot be assessed by animal testing due to either cost or recent legislation the TGx-DDI approach addresses a critical need.

What is claimed is:

1. A method of identifying a test agent as a DNA damaging agent (DDI) agent or a non-DDI agent comprising:
a) contacting a cell with a test agent;
b) lysing the cell;
c) obtaining a gene expression profile by determining the expression levels of 64 genes from Table 1 in the cell lysate, wherein the expression levels of 64 genes are determined using direct digital counting; and
d) comparing the gene expression profile obtained in step c) to a gene expression profile for each of a plurality of training samples that have been classified in a subtype of DDI agents or a subtype of non-DDI agents, wherein the gene expression profile for each of the plurality of training samples is based on expression of 64 biomarkers from Table 1, and wherein a supervised algorithm was used to construct centroids for each of the DDI and non-DDI agent subtypes in the training set;
e) calculating the distance of the gene expression profile obtained in step b) to each of the centroids; and
f) identifying the test agent as a DDI agent or a non-DDI agent based upon the nearest centroid.

2. The method of claim 1, wherein the gene expression profile obtained in step c) is compared to the gene expression profile deposited as accession number GSE58431 or the gene expression profile deposited as accession GSE107162 in the National Center for Biotechnology Information Gene Expression Omnibus.

3. The method of claim 1, wherein the cell is a p53 competent cell.

4. The method of claim 3, wherein the cell is a TK6 cell.

5. The method of claim 1, wherein the cell is contacted with the test agent in the presence of S9 rat liver extract.

6. The method of claim 1, wherein the test agent is a chemical.

7. The method of claim 6, wherein the chemical is a drug.

8. The method of claim 1, wherein a population of cells is contacted with the test agent.

9. The method of claim 1, wherein the method is a high throughput method.

10. The method of claim 1, wherein the mRNA expression levels of the 64 genes is determined.

11. The method of claim 1, further comprising isolating RNA from the cell lysate prior to determining the mRNA expression levels of 64 genes in the isolated RNA.

12. The method of claim 1, wherein the expression levels of the 64 genes are determined by:
(i) contacting the cell lysate with at least 64 probes, wherein each probe comprises a targeting sequence and a unique molecular barcode sequence and wherein the targeting domain specifically binds to a nucleotide sequence in the mRNA expressed from one of the 64 genes;
(ii) allowing the probes to hybridize to the mRNAs in the cell lysate;
(iii) directly counting the number of unique molecular barcode sequences in the probe/mRNA complexes to determine the expression levels of the 64 genes.

13. The method of claim 12, wherein the expression levels of the 64 genes are determined by:
(i) contacting the isolated RNA with at least 64 probes, wherein each probe comprises a targeting sequence and a unique molecular barcode sequence and wherein the targeting domain specifically binds to a nucleotide sequence in the mRNA expressed from one of the 64 biomarkers;
(ii) allowing the probes to hybridize to the mRNAs in the cell lysate;
(iii) directly counting the number of unique molecular barcode sequences in the probe/mRNA complexes to determine the expression levels of the 64 genes.

14. The method of claim 1, wherein data obtained from the gene expression profiles for the training samples and the gene expression profile from the cells contacted with the test agent are processed via normalization methods prior to analysis.

15. The method of claim 14, wherein said processing comprises normalization to a set of housekeeping genes.

16. The method of claim 1, wherein the test agent is an agent known to cause chromosomal damage

17. The method of claim 16, wherein the agent causes chromosomal damage in an in vitro chromosomal assay.
